(19) United States
(12) Patent Application Publication
(10) Pub. No.: US 2015/0010724 A1
(43) Pub. Date: Jan. 8, 2015

(54) FLUOROCARBON, METHOD FOR PREPARING FLUOROCARBON, AND USE THEREOF

(71) Applicant: Tokyo Ohka Kogyo Co., Ltd., Kawasaki-shi (JP)

(72) Inventors: Toshiyuki Ogata, Kawasaki-shi (JP); Yasuo Suzuki, Kawasaki-shi (JP); Atsushi Matsushita, Kawasaki-shi (JP); Tatsuhiro Mitake, Kawasaki-shi (JP)

(21) Appl. No.: 14/315,890

(22) Filed: Jun. 26, 2014

(30) Foreign Application Priority Data
Jul. 2, 2013 (JP) 2013-139165

(51) Int. Cl. C09J 7/02; C07C 22/00; C09D 7/12

(52) U.S. Cl. CPC C09J 7/0228; C09D 7/1266; C07C 22/00
USPC 428/40.7; 428/402; 106/287.27; 204/157.95

(57) ABSTRACT

A fluorocarbon including fluorine and carbon in which d50 in a cumulative particle size distribution is 1.0 nm or greater and 4.0 nm or less. A method for producing the fluorocarbon includes a plasma treatment process of generating radicals by producing inductively coupled plasma (ICP) using a fluorocarbon gas.

### CROSS-REFERENCE TO RELATED APPLICATIONS

This application claims priority under 35 U.S.C. §119(a)-(d) to Japanese Patent Application No. 2013-139165, filed Jul. 2, 2013, the entire content of which is incorporated herein by reference.

### BACKGROUND OF THE INVENTION

1. Field of the Invention

The present invention relates to a novel fluorocarbon.

2. Background Art

In recent years, a fluorocarbon has been used in various uses such as a water repellent material, a solid lubricant, and an electrode active material based on extremely low surface free energy and electrochemical properties thereof. For example, JP-A-2006-274322 discloses a technique in which a water repellent treatment is performed on an object to be treated by depositing a fluorocarbon on the object to be treated.

### SUMMARY OF THE INVENTION

As described above, the fluorocarbon has been used in various uses. For this reason, it is very useful to provide a novel fluorocarbon having properties different from those in the fluorocarbon in the related art.

The present invention has been made in consideration of the above problems, and a main object of the present invention is to provide a novel fluorocarbon, a preparation method thereof, and a use thereof.

In order to solve the above problems, the fluorocarbon according to the present invention consists of fluorine and carbon, and d50 in the cumulative particle size distribution is 1.0 nm or greater and 4.0 nm or less.

According to the present invention, it is possible to provide a novel fluorocarbon, a preparation method thereof, and a use thereof.

### BRIEF DESCRIPTION OF THE DRAWINGS

FIG. 1 is a graph showing a measurement result obtained from an infrared absorption spectrum of a fluorocarbon in Example.

FIG. 2 is a cross-sectional view schematically showing a schematic configuration of a plasma treatment apparatus used in Example.

FIG. 3 is a cross-sectional view schematically showing a schematic configuration of other plasma treatment apparatus used in Example.

### DETAILED DESCRIPTION OF THE INVENTION

Fluorocarbon

The present inventors have developed laminates for temporarily supporting a substrate in a semiconductor microfabrication process, found that a release layer manufactured by their own method consists of a novel fluorocarbon when carrying out the improvement of the release layer included in the laminate, and completed the present invention.

Hereinafter, the fluorocarbon according to an embodiment of the present invention will be described.

In one aspect, the fluorocarbon according to the present embodiment is a novel fluorocarbon which consists of fluorine and carbon, and of which d50 in the cumulative particle size distribution is 1.0 nm or greater and 4.0 nm or less.

On the other hand, the particle size of the fluorocarbon in the related art is larger than the particle size of the fluorocarbon according to the present embodiment. For example, the particle size of the fluorocarbon disclosed in JP-A-2006-274322 is in a range of several tens of nm to several hundreds of nm.

In addition, in one aspect, d90 in the cumulative particle size distribution of the fluorocarbon according to the present embodiment is preferably 3.0 nm or greater and 10.0 nm or less, and more preferably 3.0 nm or greater and 5.0 nm or less.

Moreover, the cumulative particle size distribution of the fluorocarbons refers to a measured value on a volume basis measured by determining an autocorrelation function by analyzing scattered light from particles by a dynamic light scattering method using a photon correlation method with respect to a dispersion of the fluorocarbons using a particle size measuring apparatus (SZ-100-S, manufactured by Horiba, Ltd.).

In addition, in one aspect, in the fluorocarbon according to the present embodiment, a composition ratio F/C between fluorine and carbon is preferably 0.35 or greater and 0.60 or less. Moreover, the composition ratio F/C between fluorine and carbon in the fluorocarbon refers to a value obtained by elemental analysis of the fluorocarbon.

On the other hand, the composition ratio F/C in the fluorocarbon disclosed in JP-A-2006-274322 is in a range of 1 to 2. Therefore, a ratio of carbon in the fluorocarbon according to the present embodiment is greater than that in the fluorocarbon in the related art.

In addition, considering that the composition ratio F/C of carbon and fluorine in polytetrafluoroethylene (PTFE) having a linear chain structure is approximately 2, it is estimated that a random network of carbon is widely spread in the fluorocarbon according to the present embodiment.

Furthermore, in one aspect, the fluorocarbon according to the present embodiment preferably has at least one of a double bond and a cyclic structure. That is, the fluorocarbon according to the present embodiment preferably has a conjugated system in the structure thereof. Therefore, it is estimated that the fluorocarbon according to the present embodiment has the random network structure including a double bond of carbon or a wide conjugated system in addition to a single bond of carbon, and has a structure similar to a so called amorphous carbon.

In addition, in one aspect, in the fluorocarbon according to the present embodiment, a ratio of fluorine configuring a CF₃ group among fluorine in the fluorocarbon is preferably 40.0% or greater and 70.0% or less. Moreover, the ratio of fluorine configuring a CF₃ group refers to a value obtained by acquiring an area ratio of peaks due to the configuration of CF_{X} after performing waveform separation of the spectrum obtained by a solid state nuclear magnetic resonance spectroscopy (¹³C-NMR, ¹⁹F-NMR).

Effect

The fluorocarbon according to the present embodiment can have the following effects by having at least one of the characteristics described above.

Furthermore, in one aspect, the fluorocarbon according to the present embodiment has an excellent dispersibility, and as a result, a dispersion is not turbid, and the appearance thereof becomes transparent.

In addition, in one aspect, since a content of a CF₃ group in the fluorocarbon according to the present embodiment is high, molecules repel each other, and therefore cohesive force is reduced. For this reason, the fluorocarbon according to the present embodiment in a powder form is excellently dispersed into a solvent. Moreover, the fluorocarbon according to the present embodiment is not dispersed into water which has highest polarity, and is partially dispersed (only low molecular weight material is dispersed) into saturated hydrocarbons such as menthane which has the lowest polarity. However, the fluorocarbon according to the present embodiment is excellently dispersed into other many solvents, that is, polar solvents such as dimethyl formamide (DMF), dimethyl sulfoxide (DMSO), and N-methyl-2-pyrrolidone (NMP), and among these, in particular, NMP.

Moreover, usually, materials having a primary particle size of several nm easily aggregate, and therefore the materials are not easily dispersed into solvents. For example, even when nanomaterials such as a carbon nanotube, a fullerene are dispersed into a solvent, a large amount of dispersant is used. In contrast, although the particle size (d50) of primary particles is 1.0 nm or greater and 4.0 nm or less, which is very small, the fluorocarbon according to the present invention exhibits excellent dispersibility into a solvent without a dispersant.

In addition, in one aspect, the fluorocarbon according to the present embodiment exhibits extremely high hydrophobicity.

In addition, in one aspect, the fluorocarbon according to the present embodiment has low cohesive force, and therefore, when an aggregated state is decomposed by absorbed light energy, recombination thereof is difficult.

In addition, in one aspect, the fluorocarbon according to the present embodiment has high heat resistance.

Preparation Method

In one embodiment, the method for preparing the fluorocarbon according to the present invention includes a plasma treatment process of generating radicals by producing inductively coupled plasma (ICP) using a fluorocarbon gas. By producing the inductively coupled plasma, it is possible to easily generate high density plasma (HDP). Then, by recombining carbon species (carbon radical) which are generated by dissociation of fluorocarbon gas in the high density plasma, it is possible to prepare the fluorocarbon according to the present invention.

Hereinafter, a method for preparing the fluorocarbon according to one embodiment will be described in detail.

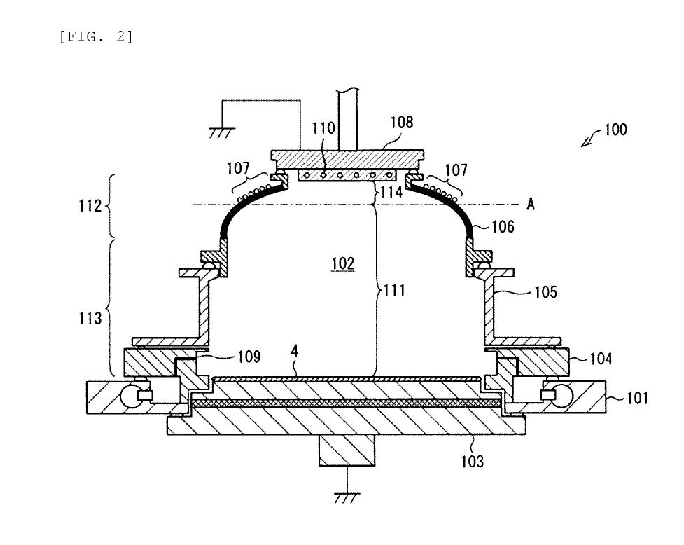

FIG. 2 is a cross-sectional view showing a configuration example of a plasma treatment apparatus used in the embodiment. Here, the structure shown in FIG. 2 is a schematic view and merely an exemplification, and the present invention is not limited thereto.

A plasma treatment apparatus 100 shown in FIG. 2 has a structure in which an exhaust ring 104 is placed on a base 101, a chamber body portion 105 is placed on the exhaust ring 104, a chamber upper portion 106 is placed onto the chamber body portion 105, a top plate 108 is overlapped on the chamber upper portion 106, and a stage 103 closes an opening below the exhaust ring 104, and configures a chamber 102 therein.

In one embodiment, the exhaust ring 104, the chamber body portion 105, and the chamber upper portion 106 are configured with quartz. On the other hand, the top plate 108 and the stage 103 are configured with metals such as aluminum and an aluminum alloy.

An upper portion of the chamber 102 is formed of a dome portion 112 having a dome shape (inverted bowl shape), and a lower portion of the chamber 102 has a shape of a bell jar type formed of a cylindrical portion 113 having a cylindrical shape. The dome portion 112 is configured with an upper portion of the chamber upper portion 106, the cylindrical portion 113 is configured with a lower portion of the chamber upper portion 106 and the chamber body portion 105.

In the exhaust ring 104, an exhaust hole 109 is provided, through which an exhaust gas is discharged from the chamber 102.

In addition, the top plate 108 is arranged so as to block an opening portion provided to a top of the dome portion 112. In the top plate 108, a supply port 110 is provided, through which a reaction gas is supplied into the chamber 102. In addition, the top plate 108 is grounded.

The stage 103 is a stage for mounting a support 4 on which the fluorocarbon is deposited, and works as a lower electrode. The stage 103 is grounded. On an outer periphery of the dome portion 112, a cap type coil 107 is arranged, and plasma is generated in a part surrounded by the cap type coil 107 in the chamber 102 (a plasma generating portion 104, a part above a straight line A in the figure).

Then, a reaction gas which is a material for forming the fluorocarbon is introduced into the chamber 102 from the supply port 110, a high frequency voltage is applied between the cap type coil 107 and the stage 103 to generate plasma, and the fluorocarbon is formed by radicals generated together with the plasma.

As a main component of the reaction gas, it is possible to use the fluorocarbon gas. Moreover, the main component refers to a gas which has the highest content (% by volume) among gases which are supplied to the chamber 102. As the fluorocarbon gas, CₓF_{y} and CₓH_{y}F_{z} are exemplified (x, y, and z are natural numbers), in more detail, CHF₃, CH₂F₂, C₂H₂F₂, C₄F₈, C₂F₆, C₅F₈ or the like are exemplified, and the fluorocarbon gas is not limited thereto.

In addition, one or more kinds of inert gases such as nitrogen, helium, argon, and the like, hydrocarbon gases such as an alkane, an alkene, an alkyne, and the like, and additive gases such as hydrogen, oxygen, and the like may be added to the reaction gas. The added amount of the additive gas is not particularly limited. For example, in a case of adding hydrogen, a ratio of 5% or greater and 20% or less with respect to the entire gas is preferably added, and the ratio is not limited thereto. In addition, the added amount of oxygen is not particularly limited, and for example, a trace amount of oxygen is added, or oxygen is not preferably added.

Moreover, the reaction gas may contain the fluorocarbon gas as a main component, and the hydrocarbon gas as an additive gas. For example, the content of additive gas with respect to the entire starting material gas is preferably 5% or greater and 20% or less. In addition, by adding an appropriate amount of inert gas to suitably stir the reactive gas, it is possible to perform uniform film-formation of the fluorocarbon on the support 4.

A flow rate of the reaction gas and a pressure in the chamber 102, which are not particularly limited, may be set to various conditions. Moreover, the reaction gas is preferably supplied from the supply port 110, and exhausted from the exhaust hole 109 by a pump or the like.

As a target temperature in the chamber 102 when performing a plasma CVD method, which is not particularly limited, a known temperature can be used, and the temperature is more preferably in a range of 100° C. to 300° C., and particularly preferably in a range of 200° C. to 250° C. By setting the temperature in the chamber 102 to such a range, it is possible to suitably perform the plasma CVD method.

In addition, the high frequency power applied to the cap type coil 107 is preferably set to be greater than the power that causes a mode jump, but the power is not limited thereto. Plasma which is a capacitive coupling subject (E mode plasma) is generally generated in a case where a plasma density is low, and plasma which is an inductive coupling subject (H mode plasma) is generally generated in a case where a plasma density is high. A transition from E mode to H mode depends on a dielectric field, and when the dielectric field becomes a certain value or higher, switching from a capacitive coupling to an inductive coupling occurs. This phenomenon is generally called “a mode jump” or “a density jump”. That is, plasma which is generated at a power not greater than the power causing the mode jump is the E mode plasma, and plasma which is generated at a power greater than the power causing the mode jump is the H mode plasma (for example, see U.S. Pat. No. 3,852,655 and U.S. Pat. No. 4,272,654). Thus, it is possible to successfully generate the high density plasma (HDP) in the plasma treatment apparatus 100.

In addition, as shown in FIG. 2, in the chamber 102 of the plasma treatment apparatus 100, a downflow region 111 is provided between the plasma generating portion 114 and the stage 103. The downflow region 111 refers to a region in which radicals generated in the plasma generating portion 114 are recombined. In the embodiment, after the radicals generated in the plasma generating portion 114 are recombined in the downflow region 111, by being deposited on the support 4, it is possible to form the fluorocarbon according to the present embodiment.

Here, in the plasma CVD apparatus in the related art, in order to improve a film-forming rate, a distance between the plasma generating portion 114 and the stage 103 is shortened. Then, by applying a cathode bias, unwanted deposits are eliminated. However, according to the findings of the present inventors, in the case of using such a plasma CVD apparatus, it is difficult to form the fluorocarbon having high light absorption properties. For example, in the case of using C₄F₈ as a reaction gas, CF₂ is produced in the plasma generating portion 114, and when this is deposited as it is, a transparent film is formed.

On the other hand, the plasma treatment apparatus 100 used in the embodiment, after the radicals generated in the plasma generating portion 114 are recombined, by being deposited on the support 4, it is possible to form the fluorocarbon according to the present embodiment. In particular, a product obtained by the recombination of carbon radicals generated in the plasma generating portion 114 has a double bond, and therefore, light absorption properties thereof are high. As a suitable example, the formed fluorocarbon may be particles of which both ends are fluorine-rich, and a central portion is carbon-rich.

The height of the downflow region 111, that is, the distance from a lower end of the plasma generating portion 114 (a lower end of the cap type coil 107) to an upper surface of the stage 103, which is not particularly limited, may be set to a distance at which the carbon radicals or the like described above can be suitably recombined. In one embodiment, the height of the downflow region 111 can be preferably set to 10 cm or greater and 20 cm or less, more preferably set to 10 cm or greater to 15 cm or less.

As described above, the top plate 108 used in the embodiment is configured with a metal, in particular, aluminum (including aluminum alloys), which is a material different from quartz configuring the adjacent chamber upper portion 106 or the like.

In the plasma treatment apparatus according to the related art, such a configuration is avoided. This is because packing of an O-ring or the like is required to connect a metal and quartz, and therefore contamination caused by the packing may occur.

However, in the embodiment, since the top plate 108 is configured with a metal, in particular, aluminum (including aluminum alloys), it is possible to cause the mode jump at a lower power, and generate inductively coupled plasma.

As described above, two types of plasma, that is, capacitively coupled plasma and inductively coupled plasma are present, and by using the inductively coupled plasma, it is possible to generate high density plasma. In particular, according to the findings of the present inventors, by using the high density plasma, it is possible to increase the generation amount of carbon radicals. Therefore, by generating inductively coupled plasma, it is possible to successfully form the fluorocarbon according to the present embodiment which is formed by recombination of carbon radicals.

In particular, by grounding the top plate 108, it is possible to reduce the power required for the mode jump. Thus, it is possible to reduce the cost of power supply equipment, power consumption, or the like.

In addition, in the embodiment, by providing the supply port 110 in the top plate 108, it is possible to supply a reaction gas from the upper side of the plasma generating portion 114, and since radicals suitably flow to the downflow region 111, it is possible to excellently deposit the fluorocarbon on the support 4.

In addition, as shown in FIG. 2, in the plasma treatment apparatus 100 used in the embodiment, the cap type coil 107 is provided on the outer periphery of the dome portion 112. That is, the cap type coil 107 is configured such that the diameter thereof is gradually increased. Thus, it is possible to reduce a resistance component in the cap type coil 107 when supplying high frequency power. From a different point of view, it is possible to increase the number of turns of the cap type coil 107 without increasing the resistance component.

The cap type coil 107 is configured with a double coil. Thus, it is possible to improve uniformity of plasma on the plane. In addition, both coils are arranged such that end portions thereof do not overlap each other, and in particular, the end portions thereof are preferably arranged at the position of line symmetry with respect to each other. Thus, it is possible to further improve uniformity of plasma on the plane.

Moreover, in the plasma treatment apparatus, when increasing the temperature of a substrate for depositing the fluorocarbon, it is possible to proportionally improve heat resistance of the fluorocarbon to be obtained. The reason therefor is as follows. A low molecular weight component which is volatilized or decomposed at the temperature of the substrate does not remain on the substrate but is discharged from the exhaust hole 109, and therefore, it is possible to leave only the fluorocarbon having high heat resistance on the support 4.

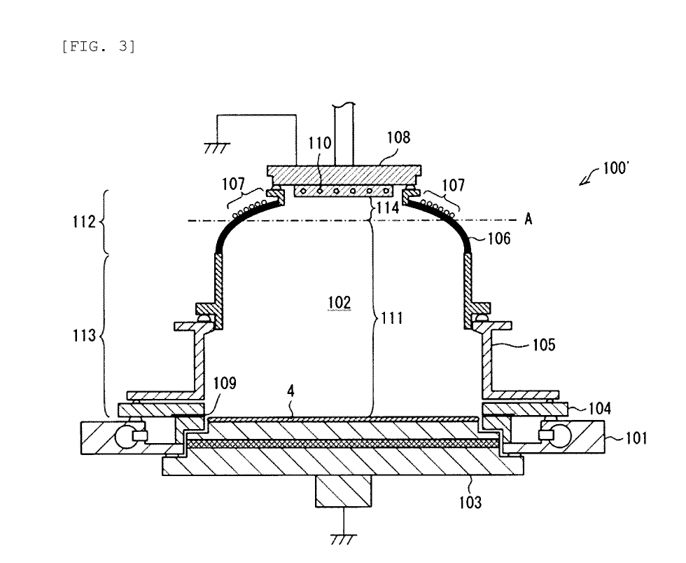

In addition, the position of the exhaust hole 109 is not particularly limited, and as the plasma treatment apparatus 100′ shown in FIG. 3, the exhaust hole 109 can also be provided at the same height as the support 4.

Since the fluorocarbon deposited on the support 4 is in an aggregated state, by disaggregating by laser irradiation or a mechanical method, it is possible to make the fluorocarbon powder. Thus, it is possible to use the fluorocarbon according to the present embodiment in various uses. Moreover, in the present embodiment, a member for depositing the fluorocarbon is not limited to the support 4.

Laminate

The uses of the fluorocarbon according to the present invention are not particularly limited, and for example, the fluorocarbon can be used as a water repellent material, a solid lubricant, and an electrode active material. In one embodiment, the fluorocarbon may used as a release layer in a laminate for temporarily supporting a substrate. Hereinafter, the laminate according to one embodiment of the present invention will be described.

The laminate according to the present embodiment is formed by laminating a substrate, an adhesive layer, a release layer, a support which transmits light in this order, and the release layer is formed of the fluorocarbon according to one embodiment of the present invention. The laminate can be used for temporarily supporting a substrate when processing a substrate.

The substrate is used for processes such as thinning, implementation, and the like in a state in which the substrate is supported by a support. As the substrate, any substrate such as a semiconductor wafer substrate, a thin film substrate, a flexible substrate, and the like can be employed. Moreover, fine structures of electronic elements such as an electric circuit, and the like may be formed on the surface of the substrate on which the adhesive layer is provided.

The adhesive layer has a configuration which bonds and fixes the substrate to the support, and protects by covering the surface of the substrate. Therefore, the adhesive layer is required to have adhesive property and strength to maintain fixation of the substrate with respect to the support and coating of the surface to be protected of the substrate when processing or transferring the substrate. On the other hand, when fixation of the substrate with respect to the support is no longer needed, the adhesive layer is required to be easily peeled off or removed from the substrate.

For example, an adhesive configuring the adhesive layer may include a thermoplastic resin in which thermal fluidity is improved by heating as an adhesive material. Examples of the thermoplastic resin include an acryl-based resin, a styrene-based resin, a maleimide-based resin, a hydrocarbon-based resin, and an elastomer.

The support has optical transparency. Therefore, when light irradiation is performed from the outside of the laminate toward the support, the light passes through the support and reaches the release layer. In addition, the support is not required to transmit all light as long as the support can transmit the light (having a predetermined wavelength) to be absorbed to the release layer.

In addition, the support is for supporting the substrate, and at the time of processes such as thinning, transfer, implementation of the substrate, the support may have strength required in order to prevent damage or deformation of the substrate. From the viewpoints as described above, examples of the support include a support formed of glass, silicon, or an acrylic resin. Moreover, the support is also referred to as a support plate.

For example, by performing (1) an adhesive layer forming step in which an adhesive layer is formed by coating an adhesive on a substrate, (2) a plasma treatment step in which a release layer is formed by forming the fluorocarbon according to the present embodiment on a support using the above-described method, and (3) an attaching step in which the substrate and the support are overlapped such that the adhesive layer and the release layer face each other, and are attached by heating and pressurizing, it is possible to form the laminate, and steps for forming the laminate are not limited thereto.

Then, after (4) performing the desired processing on the substrate in a state in which the laminate is formed, that is, in a state in which the substrate is temporarily supported on the support, by performing (5) an irradiation step in which the release layer is irradiated with light (laser) through the support, (6) a releasing step in which the substrate is released from the support, and (7) a cleaning step in which the substrate and the support are cleaned, it is possible to obtain a processed substrate and a support in a reusable state.

In one aspect, since the fluorocarbon according to the present embodiment has at least one of a double bond and a cyclic structure, the fluorocarbon can successfully absorb light. In addition, in the fluorocarbon according to the present embodiment, heat resistance at a linear chain and terminals of a fluorocarbon present in a part is low, and therefore, when light irradiation is performed in the irradiation step, fine particles of aggregated fluorocarbons are scattered. Furthermore, since a content of a CF₃ group in the fluorocarbon according to the present embodiment is high, molecules repel each other, and therefore cohesive force is reduced. Therefore there is no possibility of fine particles of the fluorocarbon which were once scattered being recombined.

Thus, the release layer formed of the fluorocarbon according to the present embodiment is changed in quality by absorbing light, and as a result, the release layer loses strength or adhesive property before being irradiated with light. Therefore, by applying a slight external force (for example, lifting the support) in the releasing step, the release layer is broken, and thus it is possible to easily separate the support and the substrate.

In addition, as described above, since content of a CF₃ group in the fluorocarbon according to the present embodiment is high, and cohesive force is weak, the fluorocarbon is easily dispersed into a solvent. For this reason, in the cleaning step, it is possible to easily clean and remove the fluorocarbon remained on the substrate or the support.

In addition, the fluorocarbon according to the present invention, in one embodiment, may be used as a water repellent material for a surface treatment of a substrate. Hereinafter, another laminate according to one embodiment of the present invention will be described.

Another laminate according to one embodiment of the present invention is configured with a substrate and a film formed of the fluorocarbon according to one embodiment of the present invention. Here, the film is used to hydrophobize a surface of a substrate.

That is, as one example, the laminate according to the embodiment is used to form a desired element on a surface of a substrate. Here, a surface treatment film is formed for a pre-processing to form an element on one surface of the substrate. In one embodiment, by forming the surface treatment film on a surface of the substrate on which an element is formed using the fluorocarbon according to the present invention as a water repellent material, it is possible to hydrophobize the surface of the substrate on which an element is formed. Thus, it is possible to improve compatibility of a photoresist and the substrate on which the surface treatment film is formed. Therefore, it is possible to uniformly coat a photoresist on the substrate, and to improve adhesiveness of the photoresist on the substrate.

The laminate according to the embodiment is manufactured by performing a surface treatment step in which a film formed of the fluorocarbon according to the present invention is formed on a substrate. Thereafter, a photoresist is coated on the substrate on which the surface treatment film was formed, and pre-baking is performed. Next, the photoresist is exposed by irradiation of ultraviolet rays, electron beams, X-ray, or the like. After exposure, a developer is coated on the photoresist, and a rinsing treatment is performed. Thereafter, it is possible to form a desired resist pattern through post-baking. Thereafter, a desired element is formed on a substrate based on the resist pattern.

The present invention is not limited thereto and can be generally used in a hydrophobization treatment of a glass substrate such as a support plate, a semiconductor substrate such as a silicon substrate, a film substrate, or the like.

Dispersion

As described above, the fluorocarbon according to the present invention exhibits excellent dispersibility in a polar solvent. Thus, in one embodiment, it is possible to use the fluorocarbon according to the present invention as a dispersion. Hereinafter, the dispersion according to one embodiment of the present invention will be described.

The dispersion according to the embodiment is obtained by dispersing the fluorocarbon according to the present invention in a solvent. Here, the dispersion disperses extremely fine fluorocarbon particles of which d50 in the cumulative particle size distribution is 1.0 nm or greater and 4.0 nm or less. That is, the fluorocarbon particles having an extremely large surface area are stably dispersed in the dispersion. Therefore, characteristics such as chemical stability, dispersion stability in a solvent, and conductivity are excellent.

Thus, in one example, the fluorocarbon according to the present invention can be used as an electrode active material which plays a central role in an oxidation-reduction reaction at an electrode in a battery. In order to use the fluorocarbon as the electrode active material, in one embodiment, the fluorocarbon can be used as a dispersion for manufacturing a current collector (electrode) having excellent conductivity. For example, the electrode active material can be used in an electrode of a lithium battery. Moreover, the electrode active material can be used for either of a positive electrode active material and a negative electrode active material depending on the type of the battery.

Examples of a dispersion medium used in the dispersion include lactones such as γ-butyrolactone and the like; ketones such as acetone, methylethylketone, cyclohexanone, methyl-n-pentylketone, methylisopentylketone, 2-heptanone, and the like; polyols such as ethyleneglycol, diethyleneglycol, propyleneglycol, dipropyleneglycol, and the like; compounds having an ester bond, such as ethyleneglycol monoacetate, diethyleneglycol monoacetate, propyleneglycol monoacetate, dipropyleneglycol monoacetate, and the like; monoalkyl ether of the polyols or the compounds having the ester bond, such as monomethyl ether, monoethyl ether, monopropyl ether, monobutyl ether; derivatives of polyols such as compounds having an ether bond, such as monophenyl ether (among these, propyleneglycol monomethyl ether acetate (PGMEA) and propyleneglycol monomethyl ether (PGME) are preferable); cyclic ethers such as tetrahydrofurane (THF) and dioxane; esters such as methyl lactate, ethyl lactate (EL), methyl acetate, ethyl acetate, butyl acetate, methyl pyruvate, ethyl pyruvate, methyl methoxypropionate, ethyl ethoxypropionate, and the like; aromatic-based organic solvents such as anisole, ethylbenzil ether, cresylmethyl ether, diphenyl ether, dibenzyl ether, phenetol, butylphenyl ether, ethylbenzene, diethylbenzene, pentylbenzene, isopropyl benzene, toluene, xylene, cymene, mesitylene, and the like; and aprotic solvents such as dimethyl sulfoxide (DMSO), dimethylformamide (DMF), N-methyl-2-pyrrolidone (NMP), and the like.

Among these, polar solvents such as dimethylformamide (DMF), dimethylsulfoxide (DMSO), N-methyl-2-pyrrolidone (NMP), THF, PGMEA, and the like are preferable.

The fluorocarbon according to the present invention is preferably dispersed in a mixing amount in a range of 0.1% by weight or greater and 20% by weight or less in the above-described solvent, and more preferably in a range of 1.0% by weight or greater and 10% by weight or less. When the mixing amount of the fluorocarbon is in the above-described range, it is possible to disperse a sufficient amount of the fluorocarbon while maintaining excellent dispersibility in a polar solvent.

In addition, various additives can be mixed with the dispersion. Examples of the additives include a dispersant, a conductive assistant, a binder, and the like. It is possible to manufacture slurry by mixing these additives with the dispersion. Next, the slurry is coated on a current collector substrate such as aluminum, and dried. Furthermore, it is possible to manufacture a current collector by compression molding of the dried slurry.

The conductive assistant is added to improve conductivity of an electrode, and examples thereof include carbon black, graphite, vapor phase grown carbon fiber, and the like.

The dispersant is an additive to prevent aggregation of the fluorocarbon according to the present invention, and examples thereof include a polymer dispersant such as polyacrylate, a salt of a copolymer of α-olefin and maleic acid, a formalin condensate of naphthalene sulfonate, polystyrene sulfonic acid, partial alkyl ester of a copolymer of styrene and maleic acid, and polyalkylene polyamine. Here, the fluorocarbon according to the present invention has excellent dispersibility in a polar solvent. Therefore, it is possible to reduce a mixing amount of the dispersant.

The binder binds the fluorocarbon, the conductive assistant, and the like such that a current collector can be formed, and examples thereof include fluorine-based polymers such as polytetrafluoroethylene and polyvinylidene fluoride, polyolefin-basedpolymers such as polyethylene, polypropylene, ethylene-propylene-diene terpolymer, and styrene-butadiene rubber.

### EXAMPLE

### Preparation of Fluorocarbon

Using a plasma treatment apparatus 100 of which a schematic configuration is shown in FIG. 2 or a plasma treatment apparatus 100′ of which a schematic configuration is schematically shown in FIG. 3, Samples 1 to 3 of a fluorocarbon were prepared. Moreover, the general conditions for preparing each sample are as follows.

When preparing Sample 1, the plasma treatment apparatus 100 of which a schematic configuration is schematically shown in FIG. 2 was used. Here, in the plasma treatment apparatus 100, an exhaust hole 109 is provided at a position vertically higher, by 16.5 mm, than an upper surface portion of a 12-inch glass substrate. In addition, as the 12-inch glass substrate used in preparation of Sample 1, a substrate of which a surface on which a fluorocarbon is formed was subjected to a hexamethylenedisilazane (HMDS) treatment was used. Moreover, in the preparation of Sample 1, the 12-inch glass substrate was installed under the condition that cleaning in the chamber 102 was not performed after performing the plasma treatment step of the previous time, and the plasma treatment step was performed.

When preparing Sample 2 and Sample 3, the plasma treatment apparatus 100′ of which a schematic configuration is schematically shown in FIG. 3 was used. Here, in the plasma treatment apparatus 100′, the exhaust hole 109 is provided at the same height as the 12-inch glass substrate. In addition, as the 12-inch glass substrate used in preparation of Sample 2 and Sample 3, a substrate of which a surface on which a fluorocarbon is formed was not subjected to a hexamethylenedisilazane (HMDS) treatment was used.

In the preparation of Sample 2, the 12-inch glass substrate was installed under the condition that cleaning in the chamber 102 was performed, and the plasma treatment step was performed.

In the preparation of Sample 3, the 12-inch glass substrate was installed under the condition that cleaning in the chamber 102 was not performed after performing the plasma treatment step, and the plasma treatment step was performed.

The 12-inch glass substrate (thickness of 700 μm) was installed in the chamber 102 of each plasma treatment apparatus, and a plasma CVD method was performed to form a fluorocarbon film on the glass substrate while supplying C₄H₈ gas at a flow rate of 400 sccm, under the conditions of a pressure of 700 mTorr, a high frequency power of 2800 W (greater power than the power to cause a mode jump), and a film-forming temperature of 240° C. (setting value).

Among respective fluorocarbon films formed by the plasma treatment step, the fluorocarbon formed within 100 nm from the center of the 12-inch glass substrate was scraped to collect, and the collected fluorocarbon was used as Samples 1 to 3.

Moreover, during the plasma treatment, a plasma light emission spectrum was measured using a plasma monitor C10346 manufactured by Hamamatsu Photonics K.K. As a result, it was possible to observe peaks due to carbon radicals (C₁, C₂, and C₃) and peaks due to fluorine radicals.

The generation of fluorine radicals and carbon radicals (C₁, C₂, and C₃) was confirmed, and from this, it was confirmed that a C—F bond of the fluorocarbon was broken by inductively coupled radicals. In addition, carbon radicals (C₁) were confirmed, and from this, it was confirmed that C₄F₈ was decomposed to the atomic level.

Measurement of Particle Size Distribution

The particle size distributions of Samples 1 to 3 were measured by a dynamic light scattering method.

Dispersions were prepared by dispersing Samples 1 to 3 in N-methyl-2-pyrrolidone.

The particle size distributions of dispersions of Samples 1 to 3 obtained by the above were measured by the dynamic light scattering method using a particle size measuring apparatus (SZ-100-S, manufactured by Horiba Ltd.). The particle size distribution of each sample was measured three times, and an average of each sample was obtained. The measurement results are shown in Table 1.

TABLE 1

| | Sample 1 | Sample 2 | Sample 3 |
|---|---|---|---|
| Median diameter (d50) | 2.2 nm | 1.8 nm | 2.1 nm |
| Median diameter (d50) | 1.6 nm | 1.2 nm | 1.4 nm |
| Median diameter (d50) | 3.9 nm | 3.5 nm | 3.8 nm |

Table 1 shows values of d50, d10, and d90 in the cumulative particle size distribution based on volumes of Samples 1 to 3. As shown in Table 1, d50 of Samples 1 to 3 was in a range of 1.8 nm or greater and 2.2 nm or less, d10 was in a range of 1.2 nm or greater and 1.6 nm or less, and d90 was in a range of 3.5 nm or greater and 3.9 nm or less. Moreover, all dispersion of the samples had a transparent appearance without turbidity.

Analysis of Composition Ratio

A composition ratio of fluorine (F)/carbon (C) in Samples 1 to 3 was measured by organic elemental analysis.

The sample which was dried at 200° C. for 10 minutes before use was used. Each of the dried samples was accurately weighed to 0.0001 mg, and simultaneous analysis of carbon-hydrogen-nitrogen (CHN) was performed, thereby obtaining the content of carbon. In addition, the dried samples were accurately weighed to 0.001 g, and analysis by flask combustion-ion chromatography was performed, thereby obtaining the content of fluorine.

The composition ratios of Samples 1 to 3 obtained from the contents of fluorine and carbon are shown in Table 2 below.

TABLE 2

| | Sample 1 | Sample 2 | Sample 3 |
|---|---|---|---|
| Fluorine (F)/carbon C) ratio | 0.48 | 0.43 | 0.43 |

In any cases of Samples 1 to 3, the fluorine/carbon ratio was about 0.4. The composition ratio F/C of fluorine and carbon in polytetrafluoroethylene (PTFE) exemplified as an example of the fluorocarbon is approximately 2. In addition, even in the preparation of fluorocarbon by dissociation of the fluorocarbon using atmospheric pressure plasma, the fluorine/carbon ratio is 1 to 2, and therefore the composition ratio of fluorine becomes higher (JP-A-2006-274322). In contrast, here, the composition ratio of carbon in the fluorocarbon according to the present invention is high. For this reason, it is assumed that a random network of carbon is widely spread in the fluorocarbon according to the present invention from the present analysis results.

Analysis of CFₓ Configuration Ratio

Moreover, waveform separation of the spectrum of Sample obtained by a solid state nuclear magnetic resonance spectroscopy (¹⁹F-NMR) was performed, thereby obtaining an area ratio of peaks due to the configuration of CF_{X} in the fluorocarbon. The analysis results are shown in Table 3.

TABLE 3

| | CF₃ | CF₂ | CF |
|---|---|---|---|
| Composition ratio | 56.7% | 21.1% | 22.2% |

As shown in Table 3, the ratio of fluorine configuring a CF₃ group obtained from the area ratio of each peak was a high value of 56.7%. For this reason, it was confirmed that the fluorocarbon of Sample 3 has more CF₃ structure which is a terminal structure than a linear chain type structure as PTFE. In addition, since a large number of the terminal structures are present, it is assumed that the fluorocarbon according to Sample 3 has a structure having a CF₃ group all over the amorphous carbon. In the generation of the fluorocarbon using plasma in the related art, it was common that the fluorocarbon has a linear chain structure such as PTFE having a large number of CF₂ structure. However, by inductively coupled plasma, it was possible to obtain a fluorocarbon with a unique structure having a large number of a CF₃ structure which is a terminal structure by generating a fluorocarbon in which a composition ratio of carbon is high.

Infrared Ray Absorption Spectrum

A measurement of an infrared ray absorption spectrum (FT-IR spectrum measurement) was performed on Sample 1.

An FT-IR spectrum of a powdered sample was measured by an AIR method.

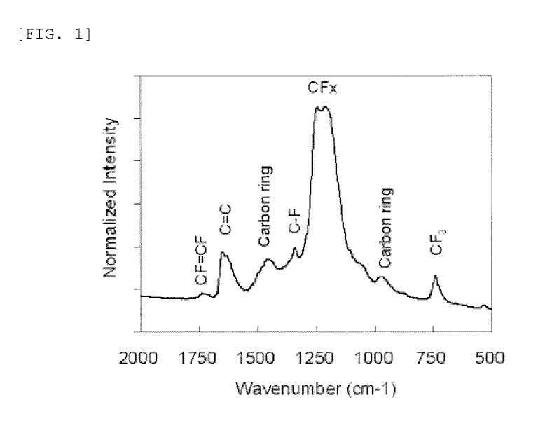

The spectrum in FIG. 1 is an FT-IR spectrum measured for Sample 1. In addition, in all of Samples 1 to 3, absorption due to a CF═CF bond was observed at 1736 cm⁻¹, absorption due to a C═C bond was observed at 1649 cm⁻¹, absorption due to a cyclic structure was observed at 1460 cm⁻¹ and 970 cm⁻¹, absorption due to a C—F bond was observed at 1342 cm⁻¹, absorption due to CF_{X} was observed at 1244 cm⁻¹ and 1209 cm⁻¹, and absorption due to CF₃ was observed at 739 cm⁻¹, respectively. In the IR data thereof, in addition to a peak of a C—F bond, data useful in management and analysis of the fluorocarbon such as peaks of a carbon double bond and a cyclic structure were obtained.

In addition, thermogravimetric analysis (TGA) of Samples 1 to 3 in a state deposited on a glass substrate was performed. As a result, in all of the samples, decomposition started from about 300° C. Also, since the samples were not completely decomposed at a temperature up to about 900° C., it is possible to assume that the samples abundantly have carbon and include a structure close to that of charcoal.

The present invention is not limited to each embodiment described above, and may be altered within the scope of the claims. That is, an embodiment derived from a proper combination of technical means disclosed in different embodiments is included in the technical scope of the present invention.

For example, the present invention can be used in all technologies in which a fluorocarbon can be used, such as a water repellent material, a solid lubricant, an electrode active material, and a release layer of laminates for temporarily supporting a substrate.

What is claimed is:

1. A fluorocarbon comprising fluorine and carbon, wherein d50 in a cumulative particle size distribution is 1.0 nm or greater and 4.0 nm or less.

2. The fluorocarbon according to claim 1, wherein d90 in the cumulative particle size distribution is 3.0 nm or greater and 10.0 nm or less.

3. The fluorocarbon according to claim 1, wherein a composition ratio F/C of the fluorine and the carbon is 0.35 or greater and 0.60 or less.

4. The fluorocarbon according to claim 2, wherein a composition ratio F/C of the fluorine and the carbon is 0.35 or greater and 0.60 or less.

5. The fluorocarbon according to claim 1, wherein a ratio of fluorine constituting a CF₃ group among fluorine contained in the fluorocarbon is 40.0% or greater and 70.0% or less.

6. The fluorocarbon according to claim 2, wherein a ratio of fluorine constituting a CF₃ group among fluorine contained in the fluorocarbon is 40.0% or greater and 70.0% or less.

7. The fluorocarbon according to claim 3, wherein a ratio of fluorine constituting a CF₃ group among fluorine contained in the fluorocarbon is 40.0% or greater and 70.0% or less.

8. The fluorocarbon according to claim 4, wherein a ratio of fluorine constituting a CF₃ group among fluorine contained in the fluorocarbon is 40.0% or greater and 70.0% or less.

9. The fluorocarbon according to claim 1 having at least one of a double bond and a cyclic structure.

10. The fluorocarbon according to claim 2 having at least one of a double bond and a cyclic structure.

11. The fluorocarbon according to claim 3 having at least one of a double bond and a cyclic structure.

12. The fluorocarbon according to claim 4 having at least one of a double bond and a cyclic structure.

13. The fluorocarbon according to claim 5 having at least one of a double bond and a cyclic structure.

14. The fluorocarbon according to claim 6 having at least one of a double bond and a cyclic structure.

15. The fluorocarbon according to claim 7 having at least one of a double bond and a cyclic structure.

16. The fluorocarbon according to claim 8 having at least one of a double bond and a cyclic structure.

17. A laminate formed by laminating a substrate, an adhesive layer, a release layer, and a support which transmits light in this order,
wherein the release layer is formed from the fluorocarbon according to claim 1.

18. A dispersion formed by dispersing the fluorocarbon according to claim 1 in a solvent.

19. A laminate comprising a substrate and a film formed from the fluorocarbon according to claim 1.

20. A method for preparing the fluorocarbon according to claim 1, comprising a plasma treatment process of generating radicals by producing inductively coupled plasma (ICP) using a fluorocarbon gas.
